(19) United States
(12) Patent Application Publication
(10) Pub. No.: US 2012/0197345 A1
(43) Pub. Date: Aug. 2, 2012

(54) Medical Device User Interface

(75) Inventor: Erwin Staller, Innsbruck (AT)

(73) Assignee: MED-EL ELEKTROMEDIZINISCHE GERAETE GMBH, Innsbruck (AT)

(21) Appl. No.: 13/358,736

(22) Filed: Jan. 26, 2012

Related U.S. Application Data
(60) Provisional application No. 61/437,304, filed on Jan. 28, 2011.

(51) Int. Cl. A61N 1/36

(52) U.S. Cl. 607/57

(57) ABSTRACT

A medical device includes a housing. A sensor, such as an accelerometer, a magnetic field sensor such as a Hall sensor, and/or a gyroscope, provides a sensor signal in response to a translation of the housing and/or a rotation of the housing. A controller controls the medical device, based at least in part, on the sensor signal.

### CROSS REFERENCE TO RELATED APPLICATIONS

This application claims priority from U.S. provisional patent application Seri. No. 61/437,304 filed Jan. 28, 2011, entitled “Medical Device User Interface,” which is hereby incorporated herein by reference it its entirety.

### TECHNICAL FIELD

The present invention relates to a device user interface, and more particularly, to a user interface for a medical device.

### BACKGROUND ART

As technology advances, external wearable medical devices are progressively getting smaller. This in turn has limited the dimensions of user interfaces associated with these devices, almost to the point below which would be considered unusable or technologically unavailable. For example, medical devices having a small housing size, such as a hearing aid, often include directly integrated buttons, trimmers, sliders, and/or switches that are already one to three millimeters in size. FIG. 1 (prior art) shows an exemplary external processor 100 associated with a middle ear implant. The external processor 100 includes a pushbutton 102 that acts as a user interface for controlling the external processor 100.

Patients have to be able to operate these devices in a safe and straightforward way. Some patients may be elderly and/or clumsy, and thus have problems to operate even normal-sized user interfaces. Operation of user interfaces may become even more difficult if the patient is under stress. Additionally, other factors such as clothes and hair may also disturb access to the user interface, especially if it is of small size.

Furthermore, medical devices often require a hermetically sealed housing and a certain amount of reliability. Conventional user interfaces such as switches and trimmers are often hard to seal against dirt and moisture, and are often prone to failure due to mechanical shock. The surface of a device may not even be available to the user, eliminating many types of user interfaces, as in the case for a fully implanted medical device.

While extra components, such as a remote control, have been implemented, these components may be inconvenient to carry, or are often lost.

### SUMMARY

In accordance with an embodiment of the invention, a medical device includes a housing. A sensor, such as an accelerometer, a magnetic field sensor such as a Hall sensor, and/or a gyroscope, provides a sensor signal in response to a translation of the housing and/or a rotation of the housing. A controller controls the medical device, based at least in part, on the sensor signal.

In accordance with related embodiments of the invention, the medical device may include a magnet external to the housing, wherein the sensor detects a magnetic field caused by the magnet. The controller controls the medical device based, at least in part, on the sensor signal indicative of the sensed magnetic field. The device may be a hearing implant that includes an external portion and an internal portion. The external portion includes the housing, the sensor and the controller, while the internal portion, for implantation within a user, includes the magnet. The device may be a cochlear implant, wherein the external portion includes a speech processor and the internal portion includes a stimulator having an electrode array for stimulating an acoustic nerve of the user.

In accordance with further related embodiments of the invention, the medical device may include a second sensor for providing a second sensor signal in response to a tap on an outside surface of the housing. The controller may control the medical device, based at least in part, on the sensor signal and the second signal.

In accordance with another embodiment of the invention, a method of interfacing with a medical device is provided. The medical device includes a housing. The method includes providing a sensor signal in response to a translation of the housing, and/or a rotation of the housing. The medical device is controlled, based at least in part, on the sensor signal.

In accordance with related embodiments of the invention, providing the sensor signal may include measuring an acceleration of the housing and/or measuring a magnetic field. The medical device may include a magnet external to the housing, wherein providing the sensor signal includes sensing the magnetic field caused by the magnet. The medical device may include an external portion and an internal portion, the internal portion implanted in a user, the external portion including a sensor for providing the sensor signal, the internal portion including the magnet. The medical device may be a cochlear implant, wherein the external portion includes a speech processor positioned within the housing, and wherein the internal portion includes a stimulator having an electrode array, the method further including activating an electrode in the electrode array so as to stimulate the acoustic nerve of the user.

In accordance with further related embodiments of the invention, the method includes providing a second sensor signal in response to a tap on an outside surface of the housing. The medical device may be controlled, based at least in part, on the sensor signal and the second sensor signal.

### BRIEF DESCRIPTION OF THE DRAWINGS

The foregoing features of the invention will be more readily understood by reference to the following detailed description, taken with reference to the accompanying drawings, in which:

FIG. 1 (prior art) shows an exemplary external processor associated with a middle ear implant;

FIG. 2 shows a schematic of a medical device, in accordance with an embodiment of the invention;

FIG. 3 shows a medical device which is controlled, at least in part, by a translation of the entire device, in accordance with an embodiment of the invention;

FIG. 4 shows a medical device which is controlled, at least in part, by rotation of the entire device counterclockwise, in accordance with an embodiment of the invention;

FIG. 5 shows a medical device which is controlled, at least in part, by rotation of the entire device clockwise, in accordance with an embodiment of the invention; and

FIG. 6 shows a medical device which is controlled, at least in part, by tapping, in accordance with an embodiment of the invention.

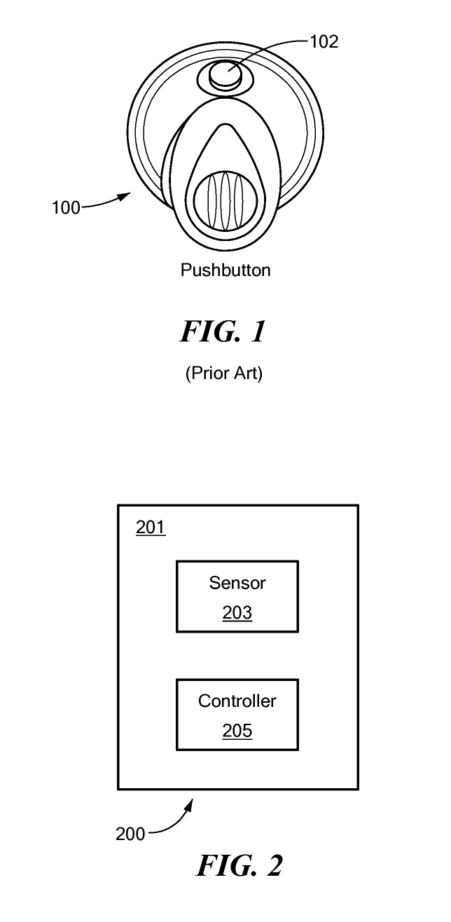

### DETAILED DESCRIPTION

In illustrative embodiments of the invention, a user interface for a medical device allows a user to control the device by translation and/or rotation of the entire device. Thus, typical Man Machine Interface (MMI) controls positioned on the outside housing of the medical device are minimized or no longer needed. Details are discussed below.

FIG. 2 shows a schematic of a medical device 200, in accordance with an embodiment of the invention. Illustratively, the medical device may be, without limitation, a hearing aid device, such as an external speech processor for a cochlear or middle ear implant.

For example, with regard to a cochlear implant, a speech processor may be positioned external to the user (for example, behind the ear), and include a microphone, a power supply (batteries) for the overall system and a processor that is used to perform signal processing of the acoustic signal to extract the stimulation parameters. The stimulation parameters are transferred via, for example, a radio frequency link, to an implanted portion that includes a stimulator having an electrode array. The stimulator generates the stimulation patterns (based on the extracted audio information) that are sent through an electrode lead to the implanted electrode array. Typically, this electrode array includes multiple electrodes on its surface that provide selective stimulation of the cochlea. For example, each electrode of the cochlear implant is often stimulated with signals within an assigned frequency band based on the organization of the inner ear. The placement of each electrode within the cochlea is typically based on its assigned frequency band, with electrodes closer to the base of the cochlea generally corresponding to higher frequency bands.

The medical device 200 includes a housing 201. A sensor(s) 203 is operatively coupled to the housing 201 and may be positioned external or internal to the housing 201. The sensor(s) 200 provides a sensor signal in response to a translation and/or rotation of the housing 201. In various embodiments, the sensor(s) may provide a sensor signal indicative of a tap on the housing 201, as described below.

The sensor(s) 203 may include, without limitation, an accelerometer, a gyroscope, a magnetic field sensor such as a Hall Sensor, and/or other sensors known in the art. The sensor(s) 203 may be a micro electro-mechanical systems (MEMS) device. The sensor(s) 203 may include, for example, piezoelectric, piezoresistive and capacitive components commonly used to convert mechanical motion into an electrical signal.

The sensor signal is received by a controller 205, which then controls the device 200, based at least in part, on the received sensor signal. The controller 205 may control, for example and without limitation, various settings (e.g., volume or tone control where the device 200 assists in hearing) or operational modes (e.g., on/off/sleep/programming modes). The controller 205 may be embodied in many different forms, including, but in no way limited to, computer program logic for use with a processor (e.g., a microprocessor, microcontroller, digital signal processor, or general purpose computer), programmable logic for use with a programmable logic device (e.g., a Field Programmable Gate Array (FPGA) or other PLD), discrete components, integrated circuitry (e.g., an Application Specific Integrated Circuit (ASIC)), or any other means including any combination thereof. Embodiments may include various components mounted as electronic components directly on a printed circuit board within the device 200.

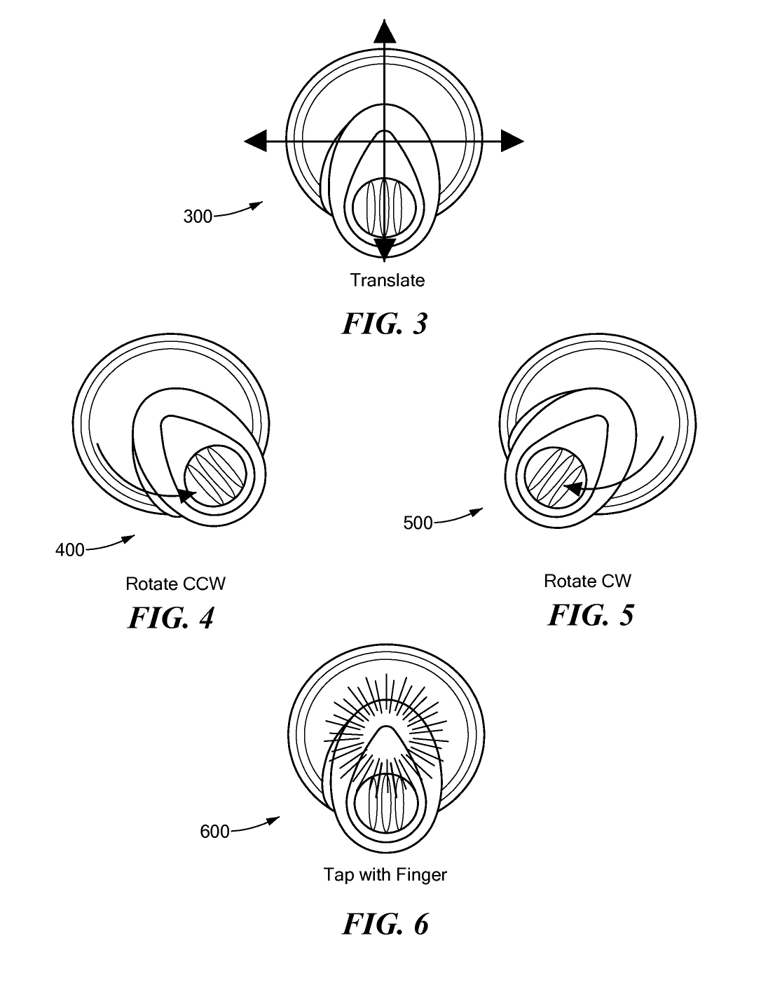

Illustratively, FIG. 3 shows a speech processor 300 which is controlled, at least in part, by a translation of the entire device 300, in accordance with an embodiment of the invention. As used in this description and the accompanying claims, the term “translation” shall mean, unless the context otherwise requires: movement without rotation. Thus, a user may control the device 300 by moving the entire device 300 left, right, up and/or down.

FIG. 4 shows a speech processor 400 which is controlled, at least in part, by rotation of the entire device 400 counterclockwise, in accordance with an embodiment of the invention, while FIG. 5 shows a speech processor 500 which is controlled, at least in part, by rotation of the entire device 500 clockwise, in accordance with an embodiment of the invention. Of course, it is to be understood that a single device may be controlled by any combination of translation and/or rotation. For example, turning the device left or right may adjust volume up or down, respectively, while moving the device minimally left/right/up/down may change other settings.

In various embodiments of the invention, the device 200 may require the user to perform a plurality of user interface actions prior to controller 205 controlling the device 200 based on the received sensor signal. For example, when the device 200, such as a speech processor, is worn by a user, any movement by the user in his/her daily routine may be sensed by the sensor 200. It may thus be important to recognize/confirm only those motions which the user intends to control device 200.

User interface actions/confirmation may be provided, without limitation, by the user translating and/or rotating the device in a predetermined manner (similar to a password). For example, a user may provide confirmation by rotating or translating the device 200 a predefined number of times in one or more various directions. The confirmation provided may be a function of time. For example, the motions required for confirmation may have to be performed in, without limitation, within three seconds.

Confirmation may be provided by tapping on the device 200, which is then sensed by sensor 203. FIG. 6 shows a medical device 600 which is controlled, at least in part, by shocks caused by tapping, in accordance with an embodiment of the invention. Illustratively, three taps on the device 600 and then turning the device 600 to the left may mean increase volume. Another tap may then confirm the setting.

In accordance with illustrative embodiments of the invention, the sensor 203 may detect a magnetic field caused by a magnet positioned external to the housing. The controller 205 controls the medical device 200 based, at least in part, on the sensor signal indicative of the sensed magnetic field. The sensor 203 may be, without limitation, a Hall Sensor.

For example, the medical device may be, without limitation, part of a cochlear implant system that includes a speech processor held in place behind the ear of the user by a magnet within an embedded stimulator. The speech processor may include a sensor that detects the magnetic field generated by the magnet within the stimulator. Any translatory or rotational movement of the speech processor relative to the magnet is sensed by the sensor. In this manner, deliberate user interface actions resulting in motions of the speech processor relative to the stimulator will advantageously be sensed by the sensor, while normal daily activities/motions by the user will generally not affect the position of the speech processor relative to the magnet, and will not be sensed.

Generally, the above-described embodiments of the invention advantageously require fewer or no external user interface components. As a result, the device may require fewer parts, reducing cost, and have minimum or no openings for dust, humidity, etc . . . Various embodiments may be completely mountable on printed circuit board. The above-described user interface will generally be advantageous compared to the smaller and generally clumsier knobs/controls on today's miniaturized devices.

Embodiments of the invention may be implemented in whole or in part in any conventional computer programming language. For example, preferred embodiments may be implemented in a procedural programming language (e.g., “C”) or an object oriented programming language (e.g., “C++”, Python). Alternative embodiments of the invention may be implemented as pre-programmed hardware elements, other related components, or as a combination of hardware and software components.

Embodiments can be implemented in whole or in part as a computer program product for use with a computer system. Such implementation may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or analog communications lines) or a medium implemented with wireless techniques (e.g., microwave, infrared or other transmission techniques). The series of computer instructions embodies all or part of the functionality previously described herein with respect to the system. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). Of course, some embodiments of the invention may be implemented as a combination of both software (e.g., a computer program product) and hardware. Still other embodiments of the invention are implemented as entirely hardware, or entirely software (e.g., a computer program product).

Although various exemplary embodiments of the invention have been disclosed, it should be apparent to those skilled in the art that various changes and modifications can be made which will achieve some of the advantages of the invention without departing from the true scope of the invention.

What is claimed is:

1. A medical device comprising:
a housing;
a sensor for providing a sensor signal in response to at least one of a translation of the housing and a rotation of the housing; and
a controller for controlling the medical device, based at least in part, on the sensor signal.

2. The medical device according to claim 1, wherein the sensor includes at least one of an accelerometer, a magnetic field sensor, and a gyroscope.

3. The medical device according to claim 1, further comprising a magnet external to the housing, wherein the sensor detects a magnetic field caused by the magnet, the controller controlling the medical device based, at least in part, on the sensor signal indicative of the sensed magnetic field.

4. The medical device according to claim 3, wherein the sensor is a magnetic field sensor.

5. The medical device according to claim 3, wherein the device is a hearing implant including an external portion and an internal portion, the external portion including the housing, the sensor and the controller, the internal portion for implantation within a user, the internal portion including the magnet.

6. The medical device according to claim 5, wherein the device is a cochlear implant, wherein the external portion includes a speech processor and the internal portion includes a stimulator having an electrode array for stimulating an acoustic nerve of the user.

7. The medical device according to claim 1, further comprising a second sensor for providing a second sensor signal in response to a tap on an outside surface of the housing.

8. The medical device according to claim 7, wherein the controller controls the medical device, based at least in part, on the sensor signal and the second signal.

9. A method of interfacing with a medical device, the medical device including a housing, the method comprising:
providing a sensor signal in response to at least one of a translation of the housing and a rotation of the housing; and
controlling the medical device, based at least in part, on the sensor signal.

10. The method according to claim 9, wherein providing the sensor signal includes measuring an acceleration of the housing.

11. The method according to claim 9, wherein providing the sensor signal includes measuring a magnetic field.

12. The method according to claim 11, wherein the medical device includes a magnet external to the housing, and wherein providing the sensor signal includes sensing the magnetic field caused by the magnet.

13. The method according to claim 12, wherein the medical device includes an external portion and an internal portion, the internal portion implanted in a user, the external portion includes a sensor for providing the sensor signal, the internal portion including the magnet.

14. The method according to claim 13, wherein the medical device is a cochlear implant, wherein the external portion includes a speech processor positioned within the housing, and wherein the internal portion includes a stimulator having an electrode array, and wherein the method includes activating an electrode in the electrode array so as to stimulate the acoustic nerve of the user.

15. The method according to claim 9, further comprising:
providing a second sensor signal in response to a tap on an outside surface of the housing; and
controlling the medical device, based at least in part, on the sensor signal and the second sensor signal.
